(19) United States
(12) Patent Application Publication
(10) Pub. No.: US 2024/0139080 A1
(43) Pub. Date: May 2, 2024

(54) TOPICAL SKIN TREATMENT

(71) Applicant: Kurt H. Ruppman, Allen, TX (US)

(72) Inventor: Kurt H. Ruppman, Allen, TX (US)

(21) Appl. No.: 18/461,486

(22) Filed: Sep. 5, 2023

Related U.S. Application Data
(60) Provisional application No. 63/403,486, filed on Oct. 18, 2022.

(51) Int. Cl. A61K 8/04; A61K 8/19; A61K 8/27; A61K 8/365; A61K 8/44; A61K 8/49; A61K 8/65; A61K 8/67; A61K 9/00; A61K 31/194; A61K 31/197; A61K 31/375; A61K 31/522; A61K 33/14; A61K 33/30; A61K 38/39; A61P 29/00; A61Q 19/02

(52) U.S. Cl. CPC A61K 8/046; A61K 8/19; A61K 8/27; A61K 8/365; A61K 8/44; A61K 8/4953; A61K 8/65; A61K 8/676; A61K 9/0014; A61K 31/194; A61K 31/197; A61K 31/375; A61K 31/522; A61K 33/14; A61K 33/30; A61K 38/39; A61P 29/00; A61Q 19/02

(57) ABSTRACT

In one aspect, the present disclosure relates to a composition comprising a mixture of: a nonessential amino acid; a collagen composition; a salt composition; zinc; potassium citrate; malic acid; vitamin C; potassium sorbate; caffeine; and water. In another aspect, the present disclosure relates to an aerosol mist formed subjecting the composition to electrolysis, ultrasonic vibration, or a combination thereof. In another aspect, the present disclosure relates to a method for treatment of skin disorders, joint pain, muscle pain, or a combination thereof, by topical application of the aerosol mist to an area of a human body proximate to one or more of skin disorders, joint pain, muscle pain, other ailments treatable through the epidermis skin layer, or a combination thereof.

### CROSS-REFERENCE TO RELATED APPLICATIONS

This application claims priority of U.S. Provisional Patent Application Ser. No. 63/403,486, entitled “Hydrogen Enriched Liquid Spray Mixture for Treating Skin Issues,” filed on Sep. 2, 2022, the contents of which are incorporated by reference herein in their entirety.

### FIELD OF THE INVENTION

This invention relates to compositions, methods, and devices for treatment of skin conditions or conditions within the human body treatable through its skin

### BACKGROUND OF THE INVENTION

There is a need for compositions and/or methods for treatment of skin disorders, joint pain, muscle pain, other human ailments treatable through the skin, or a combination thereof.

### SUMMARY OF THE INVENTION

The present invention relates to a composition comprising a mixture of: a nonessential amino acid; a collagen composition; a salt composition; zinc; potassium citrate; malic acid; vitamin C; potassium sorbate; caffeine; and water.

The present invention further relates to an aerosol mist formed when subjecting the composition to electrolysis, ultrasonic vibration, or a combination thereof.

The present invention further relates to a method for treatment of skin disorders, joint pain, muscle pain, other human ailments treatable through the skin, or a combination thereof, by topical application of the aerosol mist to an area of a human body proximate to one or more of skin disorders, joint pain, muscle pain, other ailments, or a combination thereof.

The foregoing has outlined rather broadly the features and technical advantages of the present invention in order that the detailed description of the invention that follows may be better understood. Additional features and advantages of the invention will be described hereinafter, which form the subject matter of the claims of the invention. It should be appreciated by those skilled in the art that the conception and specific embodiments disclosed may be readily utilized as a basis for modifying or designing other compositions and/or processes for carrying out the purposes of the present invention. It should also be realized by those skilled in the art that such equivalent constructions do not depart from the spirit and scope of the invention as set forth in the appended claims. The novel features which are believed to be characteristic of the invention, both as to its composition and method of manufacture, together with further objects and advantages will be better understood from the following description.

### DETAILED DESCRIPTION OF THE INVENTION

Illustrative embodiments of the subject matter claimed below will now be disclosed. In the interest of clarity, some features of some actual implementations may not be described in this specification. It will be appreciated that in the development of any such actual embodiments, numerous implementation-specific decisions must be made to achieve the developer's specific goals, such as compliance with system-related and business-related constraints, which will vary from one implementation to another. Moreover, it will be appreciated that such a development effort, even if complex and time-consuming, would be a routine undertaking for those of ordinary skill in the art having the benefit of this disclosure.

The words and phrases used herein should be understood and interpreted to have a meaning consistent with the understanding of those words and phrases by those skilled in the relevant art. No special definition of a term or phrase, i.e., a definition that is different from the ordinary and customary meaning as understood by those skilled in the art, is intended to be implied by consistent usage of the term or phrase herein. To the extent that a term or phrase is intended to have a special meaning, i.e., a meaning other than the broadest meaning understood by skilled artisans, such a special or clarifying definition will be expressly set forth in the specification in a definitional manner that provides the special or clarifying definition for the term or phrase.

For example, the following discussion contains a non-exhaustive list of definitions of several specific terms used in this disclosure (other terms may be defined or clarified in a definitional manner elsewhere herein). These definitions are intended to clarify the meanings of the terms used herein. It is believed that the terms are used in a manner consistent with their ordinary meaning, but the definitions are nonetheless specified here for clarity.

### Definitions

As used herein, “α-amino acid” means amino acids which have the amine group attached to the carbon atom next to the carboxyl group, or the alpha position. These are also known as 2-, alpha-, or α-amino acids and have a generic formula:

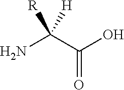

wherein R is an organic substituent (“side chain”).
   These are generally L-stereoisomers (“left-handed” isomers), while a few D-amino acids (“right-handed”) occur in bacterial envelopes and in some antibiotics. In some embodiments, the α-amino acid has a hydrophilic nonacidic side chain on the α-carbon, such as, but not limited to serine, threonine, asparagine, and glutamine. In some embodiments, the α-amino acid is one or more of serine, threonine, asparagine, and glutamine.

As used herein, “aerosol mist” means a suspension of microbubbles and/or nanobubbles of the composition disclosed herein in air. The microbubbles and/or nanobubbles are generated by application of ultrasonic vibration to the composition using devices designed to generate such aerosol mists.

As used herein, “hydrogen,” means molecular hydrogen or hydrogen not chemically bonded to any atom other than hydrogen.

As used herein, “L-arginine” means an amino acid, naturally occurring in most protein-rich foods, including fish, red meat, poultry, soy, whole grains, beans, and dairy products, and also produced naturally in the human body as a byproduct of the enzymatic production of nitric oxide from the amino acid arginine, catalyzed by nitric oxide synthase, in particular in the kidneys. Arginine is a non-essential amino acid, meaning that the body can manufacture it from other nutrients.

As used herein, “L-citrulline” means an amino acid, naturally occurring in food, such as watermelons and grapefruit, and also produced naturally in the human body as a byproduct of the enzymatic production of nitric oxide from the amino acid arginine, catalyzed by nitric oxide synthase, in particular in the kidneys. Citrulline is a non-essential amino acid, meaning that the body can manufacture it from other nutrients.

As used herein, “L-glutamine” means an α-amino acid that is used in the biosynthesis of proteins. Its side chain is similar to that of glutamic acid, except the carboxylic acid group is replaced by an amide. It is classified as a charge-neutral, polar amino acid. Glutamine is a non-essential amino acid, meaning that the body can manufacture it from other nutrients.

As used herein, “potable water” also known as drinking water, means water from surface and/or ground sources that is treated to levels that that meet state and federal standards for consumption. Potable water is water from which volatile compounds and/or solids have been removed by any suitable process, including, but not limited to, reverse osmosis and vacuum distillation.

As used herein, “treated” with respect to water, means that the water is treated by filtering, distillation, degasification, demineralization, or a combination thereof to remove volatiles, solids, or both. For example, a treated water can refer to a degassed water, a filtered water, or water that has been both filtered and degas sed.

Liquid Composition

In some embodiments, the present invention relates to a liquid composition comprising a mixture of a nonessential amino acid, a collagen composition, a salt composition, zinc, potassium citrate, malic acid, vitamin C, potassium sorbate, caffeine, and water. In some embodiments, efficacy of the composition is maintained for an extended time period by storage of the composition at or below room temperature (˜25° C.), at a temperature less than or equal to 20° C. or a temperature less than or equal to 10° C.

Nonessential Amino Acid

Nonessential amino acids are amino acids produced by the human body even if they are not ingested from food. Nonessential amino acids include and not limited to: alanine, arginine, asparagine, aspartic acid, cysteine, glutamic acid, glutamine, glycine, proline, serine, and tyrosine including all of their enantiomers such as L-tyrosine. Nonessential amino acids support tissue growth and repair, immune function, red blood cell formation, and hormone synthesis. However, unlike essential amino acids, a healthy body can create these proteins if given enough protein sources with essential amino acids.

In some embodiments, the liquid composition comprises a nonessential amino acid component, wherein the nonessential amino acid(s) comprise L-citrulline, L-arginine, L-glutamine, or a combination thereof.

In some embodiments, the nonessential amino acid(s) are present in the composition in the range of from 200 ppmw, 400 ppmw, 600 ppmw, 800 ppmw, or 1,000 ppmw to 1.5 wt %, 1.0 wt %, 0.80 wt %, 0.60 wt %, or 0.40 wt %, based on the total weight of the liquid composition.
Collagen composition

The composition disclosed herein comprises a collagen composition. The human body requires approximately 20 different amino acids in order to function properly. Preferred collagen supplements comprise a diverse amino acid profile—i.e., they contain multiple amino acids, the better. Collagen can come from many different sources, but bovine has been found to be particularly potent and effective.

In some embodiments, the collagen composition comprises type I collagen, type II collagen, type III collagen, type V collagen, type X collagen, or a combination thereof. In some embodiments, the collagen is hydrolyzed to break down the amino acids to facilitate easier absorption by the human body.

In some embodiments, the collagen composition is present in the composition in the range of from 200 ppmw, 400 ppmw, 600 ppmw, 800 ppmw, or 1,000 ppmw to 1.5 wt %, 1.0 wt %, 0.80 wt %, 0.60 wt %, or 0.40 wt %, based on the total weight of the liquid composition.

Salt Composition

The composition disclosed herein comprises a salt composition. In some embodiments, the salt composition is sodium chloride. In some embodiments, the salt composition is Dead Sea S alt.

In some embodiments, the salt composition is present in the composition in the range of from 200 ppmw, 400 ppmw, 600 ppmw, 800 ppmw, or 1,000 ppmw to 1.5 wt %, 1.0 wt %, 0.80 wt %, 0.60 wt %, or 0.40 wt %, based on the total weight of the liquid composition.

Zinc

The composition disclosed herein comprises zinc.

In some embodiments, the zinc is present in the composition in the range of from 200 ppmw, 400 ppmw, 600 ppmw, 800 ppmw, or 1,000 ppmw to 1.5 wt %, 1.0 wt %, 0.80 wt %, 0.60 wt %, or 0.40 wt %, based on the total weight of the liquid composition.

Potassium Citrate

The composition disclosed herein comprises potassium citrate.

In some embodiments, potassium citrate is present in the composition in the range of from 200 ppmw, 400 ppmw, 600 ppmw, 800 ppmw, or 1,000 ppmw to 1.5 wt %, 1.0 wt %, 0.80 wt %, 0.60 wt %, or 0.40 wt %, based on the total weight of the liquid composition.

Dicarboxylic Acid

The composition disclosed herein comprises dicarboxylic acid. In some embodiments, the dicarboxylic acid is one or more of adipic acid, fumaric acid, and malic acid. In some embodiments, the dicarboxylic acid is malic acid.

Malic acid is a natural supplement found in fruits like apples that promotes the creation of energy for human cells. Malic acid is an organic compound with the molecular formula C₄H₆O₅. It is a dicarboxylic acid that is made by all living organisms, contributes to the sour taste of fruits, and is used as a food additive. Malic acid has two stereoisomeric forms, though only the L-isomer exists naturally.

In some embodiments, the carboxylic acid is present in the composition in the range of from 200 ppmw, 400 ppmw, 600 ppmw, 800 ppmw, or 1,000 ppmw to 1.5 wt %, 1.0 wt %, 0.80 wt %, 0.60 wt %, or 0.40 wt %, based on the total weight of the liquid composition.

Vitamin C

The composition disclosed herein comprises vitamin C.

In some embodiments, vitamin C is present in the composition in the range of from 200 ppmw, 400 ppmw, 600 ppmw, 800 ppmw, or 1,000 ppmw to 1.5 wt %, 1.0 wt %, 0.80 wt %, 0.60 wt %, or 0.40 wt %, based on the total weight of the liquid composition.

Potassium Sorbate

The composition disclosed herein comprises potassium sorbate.

In some embodiments, potassium sorbate is present in the composition in the range of from 200 ppmw, 400 ppmw, 600 ppmw, 800 ppmw, or 1,000 ppmw to 1.5 wt %, 1.0 wt %, 0.80 wt %, 0.60 wt %, or 0.40 wt %, based on the total weight of the liquid composition.

Caffeine Composition

The composition disclosed herein comprises a caffeine composition. In some embodiments, the caffeine composition is natural green tea caffeine.

Natural green tea caffeine also contains the amino acid L-theanine, which can work synergistically with caffeine to improve brain function 95 to 200 mg. According to a Journal of Food Science test of caffeine levels across commercial brands of green tea, the caffeine content in each 8 oz. cup varied by brand from 11 mg to 47 mg, and other sources record green teas that can contain upwards of 60 mg per brewed cup. Green tea may have a number of health benefits including, but not limited to, assisting in weight management, reducing skin inflammation, treating type 2 diabetes, and improving cardiovascular health. Green tea has one of the highest concentrations of antioxidants of any tea. It is naturally low in calories and contains less caffeine than black tea and coffee.

In some embodiments, the caffeine composition is present in the composition in the range of from 200 ppmw, 400 ppmw, 600 ppmw, 800 ppmw, or 1,000 ppmw to 1.5 wt %, 1.0 wt %, 0.80 wt %, 0.60 wt %, or 0.40 wt %, based on the total weight of the liquid composition.

Water

The compositions disclosed herein comprise preferably a potable water. In some embodiments, the potable fluid is water is treated by filtering, distillation, degasification, demineralization, or a combination thereof.

Degassing can be accomplished by any suitable method, such as, but not limited to, reverse osmosis and vacuum distillation. Reverse osmosis is a process by which a solvent passes through a porous membrane in the direction opposite to that for natural osmosis when subjected to a hydrostatic pressure greater than the osmotic pressure. Vacuum distillation is distillation performed under reduced pressure, which allows the purification of compounds not readily distilled at ambient pressures. Regardless of the chosen method for degassing the base potable fluid, degassed potable fluids in some embodiments have a total dissolved solids (“TDS”), as determined by measuring the electrical conductivity of the potable fluid using a common TDS probe, of less than or equal to 150 ppm, 125 ppm, 100 ppm, 75 ppm, or 50 ppm.

In some embodiments, the base potable fluid is water. After degassing, the degassed potable fluid or degassed water is infused with hydrogen. Hydrogen water is made by bubbling pure hydrogen gas into water, by using electrolysis, by using a proton exchange membrane, by reaction with elemental magnesium, or by co-feed of hydrogen and a potable fluid, such as water, through a high-pressure nozzle, wherein the water is atomized into small droplets in the presence of highly concentrated hydrogen to increase hydrogen induction into the potable fluid. In some embodiments, the potable fluid, such as degassed water, is infused with hydrogen in an amount in the range of from 0.5 ppm, 1.0 ppm, 1.5 ppm, 1.6 ppm, 1.7 ppm, 1.8 ppm, 1.9 ppm, 2.0 ppm, 2.1 ppm, 2.2 ppm, 2.3 ppm, 2.4 ppm, or 2.5 ppm to 3.0 ppm, 4.0 ppm, 5.0 ppm, or 6.0 ppm by weight, wherein any listed lower endpoint of the range can be used in conjunction with any listed upper endpoint of the range to form a unique range.

In some embodiments, the water is present in the composition in an amount greater than or equal to 60 wt %, 65 wt %, 70 wt %, 75 wt %, or 80 wt %. In some embodiments, the water is present in the composition in an amount up to and including 85 wt %, 87 wt %, 91 wt %, or 95 wt %.

Aerosol Mist

In some embodiments, an aerosol mist produced by subjecting the compositions disclosed herein to electrolysis, ultrasonic vibration, or a combination thereof.

In some embodiments, the aerosol mist further comprises molecular hydrogen, hypochlorous acid, or a combination thereof, produced by subjecting the liquid composition to electrolysis. In some embodiments, the hypochlorous acid is present in an amount less than or equal to 150 ppmw.

In some embodiments, the mist comprises microbubbles, nanobubbles, or a combination thereof, produced by subjecting the liquid composition to ultrasonic vibration.

In some embodiments, a device for converting the liquid composition to an aerosol mist for delivery to the treatment area comprises a micro/nano mist sprayer as is well known in the art. Such nano mist sprayers typically generate nano and micron size bubbles as shown in Chinese Patent Publications Nos.: CN305009664 published Jan. 22, 2019, CN305009665 published Jan. 22, 2019 and CN304526519 published Mar. 2, 2018, which are herein fully incorporated by reference. Other commercial embodiments for a nano mist sprayer are available on AMAZON include a KingSmile Nano Mist Sprayer and by Hanson Mist, Model: W-718B.

The water in the formulation, in combination with a salt, undergoes electrolysis converting a portion of the water into hydrogen and in some embodiment's hydrogen and hypochlorous acid. The hydrogen gas along with the remainder of the formulation is then discharged from the nano mist sprayer as directed by the user to the preferred area of treatment.

Topical Application

Another aspect of the invention provides a method for treatment of skin disorders, joint pain, muscle pain, other ailments treatable through human epidermis, or a combination thereof. The method comprising topical application of the aerosol mist to an area of a human body proximate to one or more of skin disorders, joint pain, muscle pain, other ailments treatable through human epidermis, or a combination thereof.

In some embodiments, skin disorders cured or mitigated through short- or long-term topical application of the aerosol mist include, but are not limited to, brown spots, shingles, squamous cell skin cancer, rosacea, burns, sunburn, and skin lesions.

In some embodiments, joint pain cured or mitigated through short- or long-term topical application of the aerosol mist include, but are not limited to, osteoarthritis, rheumatoid arthritis, psoriatic arthritis, infectious arthritis, reactive arthritis, and gout.

### Certain Embodiments

Disclosed herein are compositions, methods, and devices useful for treatment of skin disorders, joint pain, muscle pain, or a combination thereof, as are described in further detail below:

Embodiment A: A composition comprising a mixture of:
a) a nonessential amino acid;
b) a collagen composition;
c) a salt composition;
d) zinc;
e) potassium citrate;
f) dicarboxylic acid;
g) vitamin C;
h) potassium sorbate;
i) caffeine; and
j) water.

Embodiment B: The composition of embodiment A, wherein the nonessential amino acid is L-citrulline, L-arginine, L-glutamine, or a combination thereof.

Embodiment C: The composition of embodiment A or B, wherein the collagen composition comprises type I collagen, type II collagen, type III collagen, type V collagen, type X collagen, or a combination thereof.

Embodiment D: The composition of any one of embodiments A-C, wherein the collagen composition is hydrolyzed.

Embodiment E: The composition of any one of embodiments A-D, wherein the salt composition is sodium chloride.

Embodiment F: The composition of any one of embodiments A-E, wherein the salt composition is Dead Sea Salt.

Embodiment G: The composition of any one of embodiments A-F, wherein the dicarboxylic acid is malic acid.

Embodiment H: The composition of any one of embodiments A-G, wherein the water is potable water.

Embodiment I: The composition of any one of embodiments A-H, wherein the water is treated by filtering, distillation, degasification, demineralization, or a combination thereof.

Embodiment J: The composition of any one of embodiments A-I, wherein the water is present in an amount greater than or equal to 70 wt % or in the range of from 60 wt % to 90 wt %, preferably great than 80% to about 98%, and components a)-i) are each present in an amount in the range of from 200 ppmw to 1.5 wt %, wherein weight percentages are based on the weight of the composition.

Embodiment K: An aerosol mist produced by subjecting the composition of any one of embodiments A-J, wherein to electrolysis, ultrasonic vibration, or a combination thereof.

Embodiment L: The aerosol mist of embodiment K, wherein the mist further comprises molecular hydrogen, hypochlorous acid, or a combination thereof.

Embodiment M: The aerosol mist of embodiment K or L, wherein the mist comprises microbubbles, nanobubbles, or a combination thereof.

Embodiment N: The aerosol mist of any one of embodiments K-M, wherein the hypochlorous acid is present in an amount less than or equal to 150 ppmw.

Embodiment O: The method for treatment of skin disorders, joint pain, muscle pain, or a combination thereof, the method comprising topical application of the aerosol mist of any one of embodiments K-N to an area of a human body proximate to one or more of skin disorders, joint pain, muscle pain, or a combination thereof.

Embodiment P: A composition comprising a mixture of: (a) a nonessential amino acid; (b) a collagen composition; (c) a salt composition; (d) dicarboxylic acid and (e) water.

The following examples illustrate the invention; however, those skilled in the art will recognize numerous variations within the spirit of the invention and scope of the claims. To facilitate a better understanding of the present invention, the following examples of preferred embodiments are given. In no way should the following examples be read to limit, or to define, the scope of the invention.

### Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Performance Testing

Cases of brown spot discoloration of skin has been cleared up with twice daily application of the aerosol mist disclosed herein.

In a case of shingles affecting the lower extremity of an individual, application of the aerosol mist disclosed herein reduced or eliminated pain within 3 minutes and was cleared up within 4 days.

In multiple cases of psoriasis, the condition has been cleared up within 6-7 months.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the compositions, processes, and/or steps described in the specification. As one of the ordinary skill in the art will readily appreciate from the disclosure of the present invention, compositions, processes, and/or steps, presently existing or later to be developed that perform substantially the same function or achieve substantially the same result as the corresponding embodiments described herein, may be utilized according to the present invention. Accordingly, the appended claims are intended to include within their scope such compositions, processes, and/or steps.

What is claimed is:

1. A composition comprising a mixture of:
a) a nonessential amino acid;
b) a collagen composition;
c) a salt composition;
d) zinc;
e) potassium citrate;
dicarboxylic acid;
g) vitamin C;
h) potassium sorbate;
i) caffeine; and
j) water.

2. The composition of claim 1, wherein the nonessential amino acid is L-citrulline, L-arginine, L-glutamine, or a combination thereof.

3. The composition of claim 1, wherein the collagen composition comprises type I collagen, type II collagen, type III collagen, type V collagen, type X collagen, or a combination thereof.

4. The composition of claim 1, wherein the collagen composition is hydrolyzed.

5. The composition of claim 1, wherein the salt composition is sodium chloride.

6. The composition of claim 1, wherein the salt composition is Dead Sea Salt.

7. The composition of claim 1, wherein the dicarboxylic acid is malic acid.

8. The composition of claim 1, wherein the water is potable water.

9. The composition of claim 1, wherein the water is treated by filtering, distillation, degasification, demineralization, or a combination thereof.

10. The composition of claim 1, wherein the water is present in an amount greater than or equal to 70 wt % or in the range of from 60 wt % to 90 wt % and components a)-i) are each present in an amount in the range of from 200 ppmw to 1.5 wt %, wherein weight percentages are based on the weight of the composition.

11. An aerosol mist produced by subjecting the composition of claim 1 to electrolysis, ultrasonic vibration, or a combination thereof.

12. The aerosol mist of claim 11, wherein the mist further comprises molecular hydrogen, hypochlorous acid, or a combination thereof.

13. The aerosol mist of claim 11, wherein the mist comprises microbubbles, nanobubbles, or a combination thereof.

14. The aerosol mist of claim 11, wherein the hypochlorous acid is present in an amount less than or equal to 150 ppmw.

15. The method for treatment of skin disorders, joint pain, muscle pain, or a combination thereof, the method comprising topical application of the aerosol mist of claim 11 to an area of a human body proximate to one or more of skin disorders, joint pain, muscle pain, or a combination thereof.
